SEQUENCE LISTING (27 sequences)

SEQ ID NO: 1 - protein, 4 aa, Homo sapiens
RGDS

SEQ ID NO: 2 - protein, 5 aa, Artificial Sequence
  Generalized fragment of homo sapiens elastin sequence VPGXG repeated 5 times where X is an amino acid preferably I or K
VPGXG

SEQ ID NO: 3 - protein, 17 aa, Homo sapiens
TVYAVTGRGDSPASSAA

SEQ ID NO: 4 - protein, 425 aa, Artificial Sequence
  Contains human fibronectin sequence, human elastin sequence, bacteriophage T7 sequence, and synthetic sequences
MASMTGGQQMGHHHHHDDDDKLQLDASTVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIGVPLE

SEQ ID NO: 5 - protein, 11 aa, Bacteriophage T7
MASMTGGQQMG

SEQ ID NO: 6 - protein, 5 aa, Artificial Sequence
  Artificial poly-histidine tag
HHHHH

SEQ ID NO: 7 - protein, 5 aa, Artificial Sequence
  Artificial Enterokinase cleavage site
DDDDK

SEQ ID NO: 8 - protein, 17 aa, Homo sapiens
TVYAVTGRGDSPASSAA

SEQ ID NO: 9 - protein, 17 aa, Artificial Sequence
  Synthetic random sequence
TVYAVTGRDGSPASSAA

SEQ ID NO: 10 - protein, 77 aa, Artificial Sequence
  Contains human elastin sequence and synthetic sequences
VPGIGVPGIGVPGRGVPGIGVPGIGVPGIGVPGIGVPGRGVPGIGVPGIGVPGIGVPGIGVPGRGVPGIGVPGIGVP

SEQ ID NO: 11 - protein, 425 aa, Artificial Sequence
  Contains human fibronectin sequence, human elastin sequence, bacteriophage T7 sequence, and synthetic sequences
MASMTGGQQMGHHHHHDDDDKLQLDASTVYAVTGRDGSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRDGSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRDGSPASSAASAVPGIGVPGIGVPGKGVPGIGVPGIGVPGIGVPGIGVPGKGVPGIGVPGIGVPGIGVPGIGVPGKGVPGIGVPGIGVPLDASTVYAVTGRDGSPASSAASAVPGIGVPGIGVPGKGVPGIGVPGIGVPGIGVPGIGVPGKGVPGIGVPGIGVPGIGVPGIGVPGKGVPGIGVPGIGVPLE

SEQ ID NO: 12 - protein, 94 aa, Artificial Sequence
  Contains human fibronectin sequence, human elastin sequence, and synthetic sequences
TVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIG

SEQ ID NO: 13 - protein, 44 aa, Artificial Sequence
  Contains human fibronectin sequence, human elastin sequence, and synthetic sequences
TVYAVTGRGDSPASSAASAVPGIGVPGIGVPGKGVPGIGVPGIG

SEQ ID NO: 14 - protein, 144 aa, Artificial Sequence
  Contains human fibronectin sequence, human elastin sequence, and synthetic sequences
TVYAVTGRGDSPASSAASAVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIGVPGIGVPGIGVPGKGVPGIGVPGIG

SEQ ID NO: 15 - protein, 94 aa, Artificial Sequence; identical to SEQ ID NO: 12
  Contains human fibronectin sequence, human elastin sequence, and synthetic sequences

SEQ ID NO: 16 - protein, 94 aa, Artificial Sequence; identical to SEQ ID NO: 12
  Contains human fibronectin sequence, human elastin sequence, and synthetic sequences

SEQ ID NO: 17 - protein, 94 aa, Artificial Sequence; identical to SEQ ID NO: 12
  Contains human fibronectin sequence, human elastin sequence, and synthetic sequences

SEQ ID NO: 18 - protein, 94 aa, Artificial Sequence
  Contains human fibronectin sequence, human elastin sequence, and synthetic sequences
ASSAASGRGDSPASSAASAVPGIGVPGIGVPGKGVPGIGVPGIGVPGIGVPGIGVPGKGVPGIGVPGIGVPGIGVPGIGVPGKGVPGIGVPGIG

SEQ ID NO: 19 - protein, 93 aa, Artificial Sequence
  Contains human fibronectin sequence, human elastin sequence, and synthetic sequences
GGSGGSGGRGDSGGSGGSVPGIGVPGIGVPGKGVPGIGVPGIGVPGIGVPGIGVPGKGVPGIGVPGIGVPGIGVPGIGVPGKGVPGIGVPGIG

SEQ ID NO: 20 - protein, 94 aa, Artificial Sequence
  Contains human fibronectin sequence, human elastin sequence, and synthetic sequences
TVYAVTGRGDSPASSAASAVPGVGVPGVGVPGKGVPGVGVPGVGVPGVGVPGVGVPGKGVPGVGVPGVGVPGVGVPGVGVPGKGVPGVGVPGVG

SEQ ID NO: 21 - protein, 94 aa, Artificial Sequence
  Contains human fibronectin sequence, human elastin sequence, and synthetic sequences
TVYAVTGRGDSPASSAASAVPGIGVPGVGVPGKGVPGIGVPGVGVPGIGVPGVGVPGKGVPGIGVPGVGVPGIGVPGVGVPGKGVPGIGVPGVG

SEQ ID NO: 22 - protein, 94 aa, Artificial Sequence
  Contains human fibronectin sequence, human elastin sequence, and synthetic sequences
TVYAVTGRGDSPASSAASAVPGKGVPGIGVPGKGVPGIGVPGKGVPGIGVPGKGVPGIGVPGKGVPGIGVPGKGVPGIGVPGKGVPGIGVPGKG

SEQ ID NO: 23 - protein, 94 aa, Artificial Sequence
  Contains human fibronectin sequence, human elastin sequence, and synthetic sequences
TVYAVTGRGDSPASSAASAVPGIGVPGKGVPGKGVPGIGVPGKGVPGKGVPGIGVPGKGVPGKGVPGIGVPGKGVPGKGVPGIGVPGKGVPGKG

SEQ ID NO: 24 - protein, 94 aa, Artificial Sequence
  Contains human fibronectin sequence, human elastin sequence, and synthetic sequences
TVYAVTGRGDSPASSAASAVPGVGVPGKGVPGKGVPGVGVPGKGVPGKGVPGVGVPGKGVPGKGVPGVGVPGKGVPGKGVPGVGVPGKGVPGKG

SEQ ID NO: 25 - protein, 94 aa, Artificial Sequence
  Contains human fibronectin sequence, human elastin sequence, and synthetic sequences
TVYAVTGRGDSPASSAASAVPGKGVPGKGVPGKGVPGKGVPGKGVPGKGVPGKGVPGKGVPGKGVPGKGVPGKGVPGKGVPGKGVPGKGVPGKG

SEQ ID NO: 26 - protein, 94 aa, Artificial Sequence
  Contains human fibronectin sequence, human elastin sequence, and synthetic sequences
TVYAVTGRGDSPASSAASAVPGIGVPGIGVPGIGVPGIGVPGKGVPGIGVPGIGVPGIGVPGIGVPGKGVPGIGVPGIGVPGIGVPGIGVPGIG

SEQ ID NO: 27 - protein, 4 aa, Artificial Sequence
  Artificial spacer sequence
LDAS

(19) United States
(12) Patent Application Publication
(10) Pub. No.: US 2014/0348894 A1
(43) Pub. Date: Nov. 27, 2014

(54) Engineered Protein Coating for Medical Implants

(71) Applicant: The Board of Trustees of the Leland Stanford Junior University, Palo Alto, CA (US)

(72) Inventors: Jordan R. Raphel, Dedham, MA (US); Andreina Parisi-Amon, San Francisco, CA (US); Sarah C. Heilshorn, Mountain View, CA (US)

(21) Appl. No.: 14/261,805

(22) Filed: Apr. 25, 2014

Related U.S. Application Data
(60) Provisional application No. 61/816,002, filed on Apr. 25, 2013.

(51) Int. Cl. A61L 31/10; A61L 31/16; A61L 31/08

(52) U.S. Cl. CPC A61L 31/10; A61L 31/088; A61L 31/16; A61L 2420/00; A61L 2400/18
USPC 424/423; 514/16.5

(57) ABSTRACT

Engineered protein coatings are provided for medical implants to promote bone regeneration. The coating is an engineered protein containing an elastin-like structural domain (SEQ ID No: 2) and a cell-adhesive domain derived from an extended fibronectin RGD sequence. The surface of the medical implant is covalently and directly bonded to the coating via photoreactive crosslinking through an insertion and/or addition reaction. The engineered protein coating can be applied directly upon fabrication of the implant, which would eliminate applying the coating in the operating room. The engineered protein coating is also customizable and can include biologics to improve performance. Furthermore, the engineered protein coating could also be spatially patterned on the implant surface.

### STATEMENT OF GOVERNMENT SPONSORED SUPPORT

This invention was made with Government support under contract no. DMR-0846363 awarded by the National Science Foundation, and under contract no. DP2-OD-006477 and R21-AR-062359 awarded by the National Institutes of Health. The Government has certain rights in this invention.

### SEQUENCE LISTING

This application includes a sequence listing submitted in written form and in computer readable form.

### FIELD OF THE INVENTION

This invention relates to coatings for synthetic, inorganic, and/or metallic surfaces to improve cell adhesion and function on the surface.

### BACKGROUND OF THE INVENTION

Protein thin films are employed components in many biomedical systems. For example, cell-adhesive proteins films that are able to coat synthetic implants, thereby presenting a more favorable surface for in vivo interactions, have found applications in nerve guides, bone grafts, and vascular grafts. Commonly, these coatings are made from adsorbed layers of native extracellular matrix (ECM) proteins including collagen, laminin, and fibronectin or other natural biopolymers.

While the appeal of using naturally derived materials for in vivo applications is clear, there are certain key limitations affecting the efficacy of these systems. When selecting naturally derived materials, one is limited to those materials that can be harvested and purified sufficiently so as to not cause immunogenic responses post-implantation. Furthermore, although bioactive, these materials suffer from significant batch-to-batch variations in their mechanical and biochemical properties, leading to irreproducible coating performance, especially on larger scales. In addition, these films are often adsorbed coatings, which can cause irregularities in thickness and density, and hence bioactivity.

Engineered proteins harness the bioactive functionality of naturally derived materials, while overcoming their limitations through highly reproducible and modular chemical compositions, making them ideal for many biomedical applications. The present invention provides such engineered proteins for coatings in medical implants.

### SUMMARY OF THE INVENTION

The present invention provides engineered protein coatings for medical implants. Examples of medical implants are a joint replacement prosthesis, a dental implant, a bone screw, a bone interfacing device, or the like. The engineered protein coating can be directly applied after the fabrication of the medical implant and would eliminate the application of the coating in the operating room preparation. This is in contrast to, for example, the use of bone cement as a coating, which is applied in the operating room. In addition, since the size, shape and feel of the implant remains the same as current implants, no additional surgical training is needed. This is in contrast to, for example, porous and hydroxyapatite coatings. The engineered protein coating is also customizable and can include biologics to improve performance, such as attaching an antibiotic to provide localized defense against post-surgical infection. Furthermore, the engineered protein coating can also be spatially patterned on the implant surface.

In one embodiment, a bone interfacing medical implant is provided. A bone interfacing coating for promoting bone regeneration is bonded to the surface of a medical implant. The coating is an engineered protein containing an elastin-like structural domain and a cell-adhesive domain derived from an extended fibronectin RGDS sequence (SEQ ID No: 1) or RGD sequence.

The elastin-like structural domain contains a repetitive amino acid sequence of the form (VPGXG)ₙ (SEQ ID No: 2), where X is an amino acid and n is at least 5. In one example, at least 5% of X residues are K amino acids. K amino acids are used to crosslink the protein into a stable coating onto the implant surface. An example of a specific formulation is 20% K and 80% I amino acids where I is used to aid in purification of the polymer by altering the polymer solubility. The cell-adhesive domain contains at least a RGDS sequence (SEQ ID No: 1) or a RGD sequence. An example of the extended fibronectin cell-binding sequence is TVYAVTGRGDSPASSAA (SEQ ID No: 3).

The surface of the medical implant is covalently and directly bonded to the coating via photoreactive crosslinking through an insertion reaction. In one example, the surface is covalently and directly bonded to the coating via heterobifunctional, photoreactive crosslinkers that are photoactivated to present carbene or nitrene groups. In another example, the photoreactive crosslinking is through an insertion reaction of carbene groups, an insertion reaction of nitrene groups, an addition reaction with free radicals, or a multi-step insertion and addition reaction of carbene or nitrene groups. Examples of surfaces to which the coating could bond are titanium, titanium alloy, stainless steel, ceramic, tantalum, tantalum alloy, cobalt-chromium alloy, magnesium alloy, alumina, zirconia, zirconia toughened alumina, hydroxyapatite/calcium phosphates, or polyether ether ketone.

### BRIEF DESCRIPTION OF THE DRAWINGS

FIGS. 1A-C show the conjugation of a photoreactive diazirine group to an engineered ELP (SEQ ID No: 4) according to an exemplary embodiment of the invention. FIG. 1A shows a schematic of crosslinker conjugation chemistry. Primary amines within ELP (present at the N-terminus and on side chains of lysine residues) react with the NHS-ester diazirine crosslinker to form an amide bond, thereby synthesizing a diazirine-modified ELP (ELP-D). Upon exposure to ultraviolet light (UV), the diazirine group forms a highly reactive carbene intermediate, which can insert into a neighboring ELP chain or bond on the substrate surface. FIG. 1B shows conjugation efficiency as a function of crosslinker:primary amines stoichiometry. FIG. 1C shows FTIR data of characteristic diazirine peak (1460 cm⁻¹) first-order exponential decay as a function of UV exposure time, with t_{1/2}=119 seconds.

FIG. 2 shows according to an exemplary embodiment of the invention an amino acid sequence of ELP (SEQ ID No: 4). The T7-lac promoter site (SEQ ID No: 5) is used to initiate expression, the poly-histadine (His) tag (SEQ ID No: 6) is utilized for identification, and the enterokinase (EK) cleavage site (SEQ ID No: 7) can be used to remove the upstream promoter and tag regions. The bioactive site contains either an extended, cell-adhesive RGD sequence from native fibronectin (SEQ ID No: 8) or a negative-control scrambled sequence (SEQ ID No: 9) that is not cell-adhesive.

FIG. 3 shows according to an exemplary embodiment of the invention a scanning electron micrograph of an ELP-D thin film spin coated on a glass coverslip (processed at 4,000 rpm, 90 sec). After spin coating, the ELP-D thin film was photocrosslinked and dehydrated. The entire glass coverslip was fractured and mounted on a 90-degree stub. Lower magnification (left) shows the entire cross-section of the glass coverslip and ELP-D thin film. The observed coverslip thickness, ˜150 μm, matches that reported by the manufacturer (Fisher). Higher magnification (right) shows the dehydrated ELP-D coating has a thickness of ˜350 nm.

FIGS. 4A-C show the photocrosslinked ELP-D stability according to an exemplary embodiment of the invention. FIG. 4A shows fluorescent images of ELP-D (top) and ELP (bottom) scaffolds micropatterned by soft lithography to have a repetitive striped topography (50 micrometers wide, 5 micrometers depth pattern). Dried scaffolds prior to UV light exposure clearly shows the presence of topography (left), while 24 hours after UV exposure and rinsing, only the hydrated ELP-D scaffold remains. FIG. 4B shows mass of protein retained in spin coated thin films prior to UV exposure and at various times post-exposure (in solution), for ELP and ELP-D samples. FIG. 4C shows long-term, hydrated stability of spin coated ELP-D thin films processed to have different initial masses.

FIGS. 5A-D show the culture of human adipose-derived stem cells (hASC) on ELP-D thin films according to an exemplary embodiment of the invention. FIG. 5A shows percentage of adherent hASC to ELP-D (which contains a cell-adhesive RGD sequence (SEQ ID No: 4)), scrambled-ELP-D (negative control (SEQ ID No: 11)), and non-coated glass coverslips (* p<0.0001, ** p<0.02). ELP-D is the left bar of the three bars in each set of bars. Scrambled ELP-D is the middle bar of the three bars in each set of bars. Non-coated glass is the right bar of the three bars in each set of bars. FIG. 5B shows hASC spread area on ELP-D and scrambled-ELP-D thin films at 4 hours. Data shown as a box-and-whisker plot; bottom and top of the box mark the 25^{th} and 75^{th} percentiles, respectively; horizontal band in the box marks the median, and whiskers mark the 5^{th} and 95^{th} percentiles (* p<0.001). FIG. 5C shows representative hASC morphology on ELP-D (top) and scrambled-ELP-D (bottom) thin films at 4 hours after seeding, with fluorescent staining of nuclei (DAPI, white) and F-actin (phalloidin, grey). FIG. 5D shows day 6 fluorescent staining of live (calcein AM, grey) and dead (ethidium homodimer) hASCs on ELP-D and scrambled-ELP-D thin films demonstrating long-term viability of cells on ELP-D materials.

FIG. 6 shows according to an exemplary embodiment of the invention hASC metabolic activity after 6 days in culture on ELP-D, scrambled-ELP-D, or non-coated glass coverslips. Metabolism levels were quantified by measuring the fluorescence produced due to reduction of the reagent. The level of metabolic activity for each group was normalized to total cell number. As expected, cells on all substrates were found to be similarly metabolically active.

FIG. 7 shows according to an exemplary embodiment of the invention that ELP-D coatings significantly improve the adhesion of bone-forming cells (MG63 osteoblast-like cells) to Ti6Al4V titanium alloy. 24 hours post-seeding, over twice as many MG63 cells adhered to ELP-D coated Ti6Al4V substrates compared with uncoated Ti6Al4V substrates.

FIG. 8 shows according to an exemplary embodiment of the invention that ELP-D coatings significantly improve the amount of mineralization that bone-forming cells can synthesize on top of a Ti6Al4V titanium alloy substrate within two weeks. Mineralization produced by MG63 cells was quantified spectroscopically and was found to be significantly higher on ELP-D coated Ti6Al4V samples after 1, 3, and 7 days in mineralization media compared with mineralization produced by MG63 cells seeded on uncoated Ti6Al4V.

FIG. 9 shows according to an exemplary embodiment of the invention that ELP-D coated commercially-pure titanium dental screws implanted into rat femurs and removed 1 week post-surgery resulted in greater removal torque and hence faster rates of healing when compared with implanted uncoated commercially-pure titanium dental screws. ELP-D coated screws showed similar removal torque and healing 4 weeks after surgery compared with uncoated screws, demonstrating that the coating does not impede long-term healing.

FIG. 10 shows according to an exemplary embodiment of the invention that ELP-D coatings on Ti6Al4V are stable for three weeks in physiological buffer. The mass of protein in the coatings does not significantly change during rinsing times of 1 hour up to 3 weeks.

FIGS. 11A-C show according to an exemplary embodiment of the invention that ELP-D coatings on commercially-pure titanium dental screws withstand the stresses associated with implantation into (i.e. before implantation, FIG. 11A) and explantation out (FIG. 11B) of a synthetic bone mimic. The ELP-D coating is not altered by the processes and remains intact over the majority of the screw surface. FIG. 11C shows a defect in the coating to demonstrate the presence of the coating in the other images. FIGS. 11A-B show images with a 1 micrometer scale bar and FIG. 11C shows an image with a 10 micrometer scale bar.

FIGS. 12A-B show according to an exemplary embodiment of the invention that ELP-D on Ti6Al4V forms a continuous coating on top of the substrate. Some topographical features from the underlying Ti6Al4V substrate can be seen through the thin ELP-D coating. FIG. 12A shows an image with a 2 micrometer scale bar and FIG. 12B shows an image with a 1 micrometer scale bar.

FIGS. 13A-B show according to an exemplary embodiment of the invention that ELP-D coatings on Ti6Al4V allow for the adhesion and spreading of bone-forming MG63 cells on the sample surface. The spread cell morphology, along with extended filopodia, demonstrates strong adhesion of the cells to the surface. FIG. 13A shows an image with a 100 micrometer scale bar and FIG. 13B shows an image with a 20 micrometer scale bar.

### DETAILED DESCRIPTION

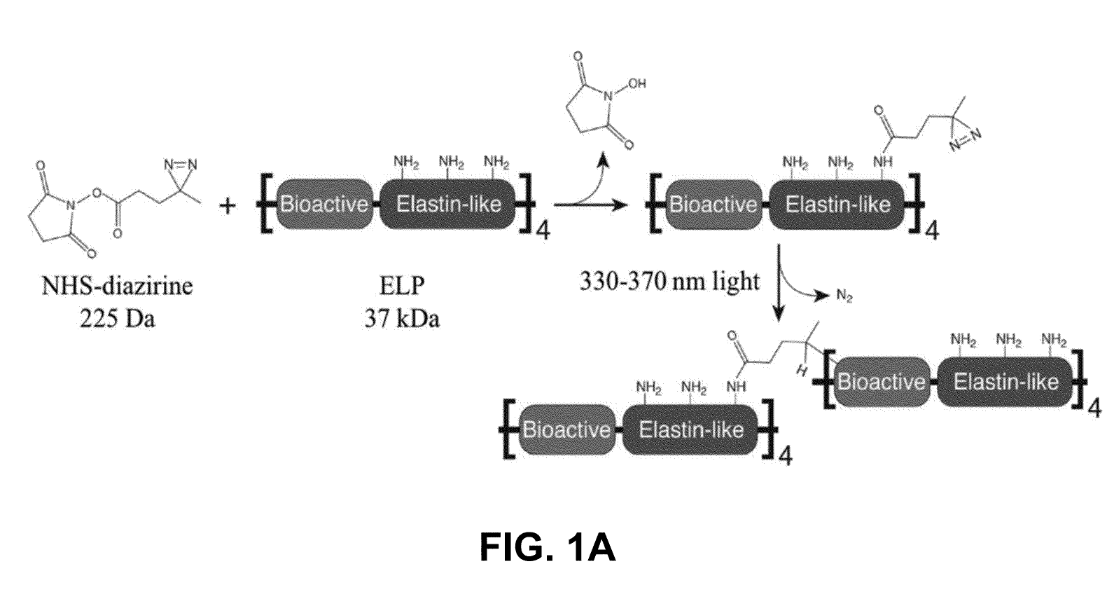

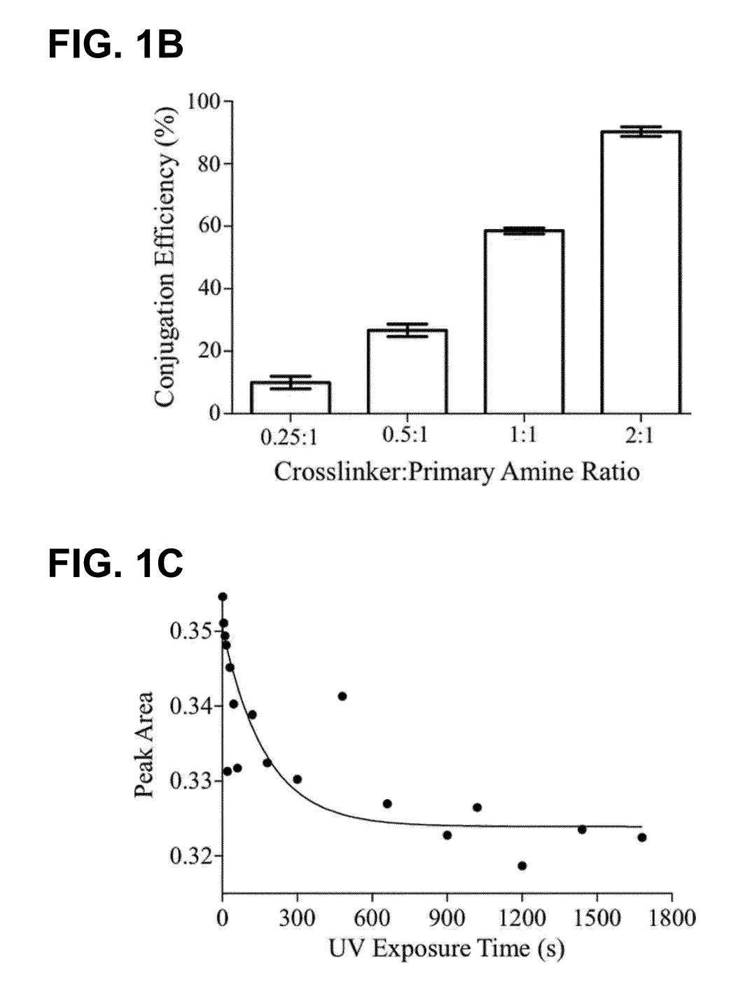

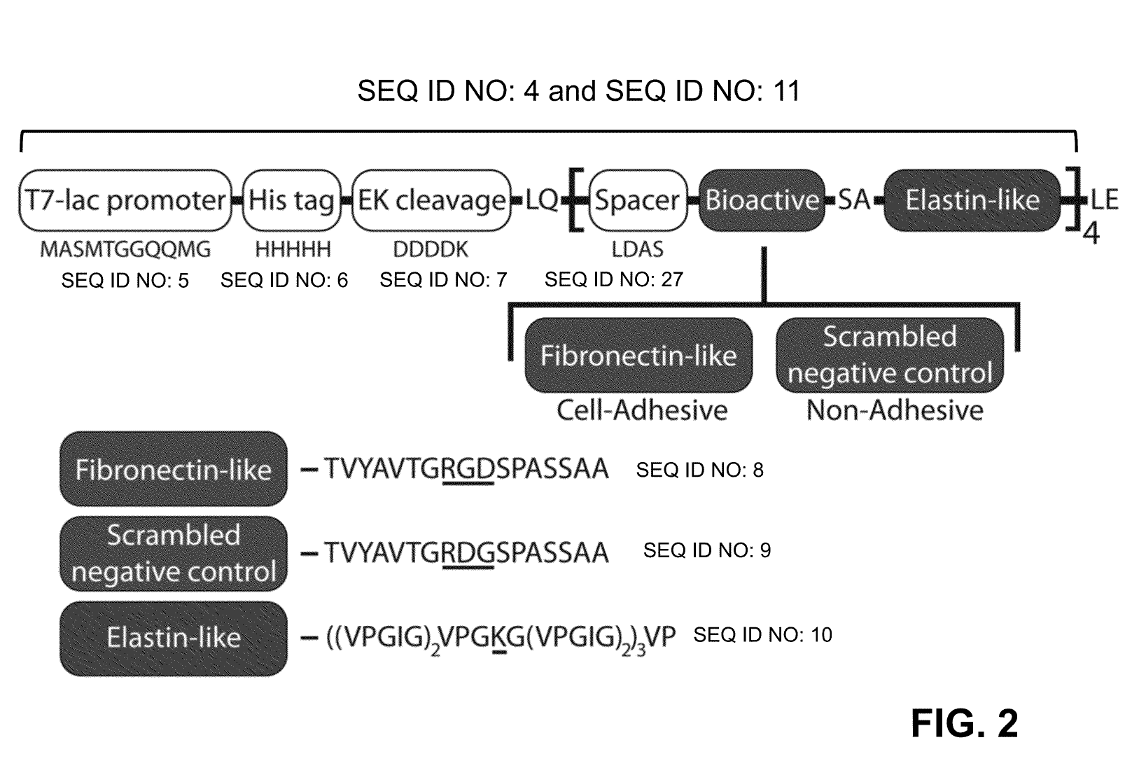

In one embodiment of the invention, we show the synthesis of an engineered elastin-like protein (ELP (SEQ ID No: 4)) in a commercially available Escherichia coli strain designed for recombinant protein yield followed by post-processing to append photoreactive moieties onto the protein backbone. The ELP used here is a block copolymer designed to contain four repeats of a peptide cassette containing an elastin-like structural domain and a bioactive, cell-adhesive domain incorporating the extended fibronectin RGD sequence (FIG. 1A, FIG. 2).

In this example, there are four total repeats of the cassette and each cassette contains 15 repeats of the basic VPGXG elastin-like sequence (SEQ ID No: 2), where X is I or K.

A heterobifunctional N-hydroxysuccinimide ester diazirine (NHS-ester diazirine, for example, succinimidyl 4,4′-azipentanoate) is conjugated to the ELP through reaction with the primary amine side chains of the canonical lysine (K) amino acid residues. Chemical modification of the engineered protein post-expression enables synthesis of a photoactive protein material while simultaneously utilizing the inherent scalability of recombinant protein expression.

We show the potential applications of this technique through synthesis of a reliable, versatile, scalable ELP biomaterial that can be photo-processed in multiple ways. Two-dimensional (2D) protein coatings are produced by spin coating and drop casting, while bulk, three-dimensional (3D) scaffolds are formed by mold casting. The coating and crosslinking procedures are reliable and straightforward, producing materials that retain their cell-adhesive biofunctionality to interact with mammalian cell types such as human adipose-derived stem cells (hASCs) and osteoblasts (bone-forming cells). Taken together, the bioactivity of this photocrosslinkable ELP material to promote cell adhesion, along with its versatility to produce coatings, thin films, and bulk scaffolds, encourage applications in implant coatings and tissue engineering scaffolds.

### Experimental

### Recombinant Synthesis and Purification of ELP

ELP (SEQ ID No: 4) and scrambled-ELP (SEQ ID No: 11) (a negative control protein containing a non-cell-adhesive sequence, FIG. 2) were expressed and purified (see Straley et al., Soft Matter, 2009 5(1):114-124). Briefly, a plasmid encoding the protein sequence was transformed into an Escherichia coli host (BL21(DE3), NEB) and expression was induced under control of the T7-lac promoter. After cell lysis, the target protein was purified by repeated centrifugation at alternating temperatures (4 degrees Celsius and 37 degrees Celsius), utilizing the lower critical solution temperature of the ELP. Protein purity was assessed by gel electrophoresis. ELP was dialyzed, lyophilized, and stored at room temperature.

### ELP-Diazirine Conjugation

A heterobifunctional N-hydroxysuccinimide ester diazirine crosslinker (NHS-ester diazirine, succinimidyl 4,4-azipentanoate, Pierce Biotechnology) was dissolved in dimethyl sulfoxide and mixed with a solution of ELP, dissolved in either 20 mM HEPES or phosphate buffered saline (PBS). After reaction, the diazirine-conjugated ELP (ELP-D) was dialyzed against deionized H₂O for 8 hours, frozen, and lyophilized. The conjugation efficiency was determined by 2,4,6-trinitrobenzene sulfonic acid (TNBSA, Pierce Biotechnology) assay to detect unreacted primary amines. Briefly, a solution of protein in phosphate buffered saline (PBS) was diluted in sodium bicarbonate buffer followed by the addition of TNBSA reagent. After two hours of incubation at room temperature, the reaction was stopped with a combination of sodium dodecyl sulfate and hydrochloric acid, and the absorbance at 335 nm was taken for comparison to a standard curve of non-conjugated ELP.

### Photocrosslinking of ELP-D Materials

ELP was resuspended from a lyophilized state in PBS at 4 degrees Celsius. All protein materials were made using 50 mg mL⁻¹ (5 wt %) solutions. Protein films were fabricated by spin coating protein solution onto 12 mm glass coverslips (Fisher). Coverslips, previously rinsed in ethanol, dried with nitrogen gas, and stored at 4 degrees Celsius for a minimum of 1 hour prior to use, were placed on the stage of a spin coater (WS-400-6NPP, Laurell Technologies). The protein solution (14 microliters) was placed onto the center of the coverslip and spun at various speeds (4000-8000 rpm) for 90 seconds. Drop cast films were made by spreading protein solution on the substrate of interest with a pipette tip. Bulk protein scaffolds were made by adding protein solution (40 microliters) to a 10 mm tall, 4.5 mm diameter circular mold. Patterned scaffolds were made with soft lithography by adding protein solution to a stripe-patterned (50 micrometers width, 5 micrometers depth) poly(dimethyl siloxane) (PDMS) mold. The solution-filled mold was covered by a glass coverslip and allowed to dry overnight at 37 degrees Celsius. After processing, all materials were exposed to ultraviolet light using a 365 nm, 8 watt light source (3UV-38, UVP). Spin coated films and PDMS patterned scaffolds were exposed for 1 hour at an exposure distance of 3 cm, while drop cast films and bulk scaffolds were exposed for 2 hours at the same conditions.

### Scanning Electron Microscopy (SEM) Characterization

SEM samples were spin coated using the protocol above, 4000 rpm for 90 seconds. Film morphology samples were left hydrated without any further processing and imaged with variable pressure SEM (Hitachi S-3400N Variable Pressure SEM, operated at 15 kV, pressure 50-60 Pa, using a Deben Coolstage for temperature control). Film thickness samples were air dried, sputter coated with a layer of gold, and imaged with field emission SEM (FEI Magellan 400 XHR, operated at 5 kV).

### Fourier Transform Infrared Spectroscopy (FTIR) Characterization

Protein films were spin coated on 13 mm diameter zinc selenide discs (Perkin Elmer) using an identical procedure as for the glass coverslips given above. Measurements were performed using an FTIR spectrometer (Vertex 70, Bruker Optics), purged with nitrogen gas. A non-coated zinc selenide disc was used for background control. The films were exposed to the 365 nm light source described above for varying amounts of time, with an FTIR measurement after each exposure time. A single measurement consisted of 200 scans with a resolution of 4 cm⁻¹. Disappearance of a characteristic diazirine peak (1460 cm⁻¹) upon photoactivation was reported as total peak area over time.

### Mechanical Properties Characterization

Uniaxial tensile tests were performed on drop cast, crosslinked ELP-D films using a mechanical testing system (Bionix 200, MTS Systems Corporation). A 44.48-N load cell was used to characterize the force-displacement curve, which was converted to engineering stress and strain using the initial dimensions of the ELP-D films. The Young's modulus is the slope of the linear portion of the engineering stress-strain curve.

### Thin Film Mass Characterization

The mass of protein in spin coated films was determined by a bicinchoninic acid (BCA) endpoint assay (QuantiPro, Sigma Aldrich). Briefly, protein films for each time point (n=4) were submerged in 500 microliters PBS, followed by the addition of 500 microliters of BCA reagent (25:25:1 QA buffer:QB buffer:copper II sulfate solution). The reaction was incubated at 60 degrees Celsius for 1 hour, equilibrated at room temperature for 20 minutes, and quantified by absorbance at 562 nm for comparison to a standard curve.

hASC Isolation and In Vitro Culture Maintenance

ASCs were isolated from human lipoaspirate from the flank and thigh regions by suction assisted liposuction. Specimens were washed in dilute betadine, rinsed twice in PBS, and digested with 0.075% Type II collagenase in Hank's Balanced Salt Solution at 37 degrees Celsius under agitation for 30 minutes. Next, collagenase was inactivated by an equal volume of PBS with 10% fetal bovine serum (FBS) and 100 IU mL⁻¹ penicillin-streptomycin. The stromal vascular fraction was then pelleted, resuspended, and filtered through a 100 micrometer strainer before being plated into a 100 mm dish. Adherent cells were cultured in DMEM supplemented with 10% FBS and 100 IU mL⁻¹ penicillin-streptomycin at 37 degrees Celsius and 5% atmospheric CO₂. Cells were expanded and passaged by trypsinization for subsequent use in in vitro assays.

hASC Culture on Photocrosslinked ELP-D Thin Films

Cells were seeded at 1.24×10⁴ cells cm⁻² onto spin coated thin films of ELP-diazirine (ELP-D), scrambled-ELP-D, or non-coated 12 mm glass coverslips (Fisher) (n=4 independent samples for each condition). Phase contrast images were taken at 2, 3, 4, 5, and 6 hours post-seeding using an inverted light microscope (Zeiss Axiovert) at four random positions for each substrate. Individual cells were scored as either adherent (i.e., appearing dark by phase contrast) or non-adherent (i.e., appearing refractile by phase contrast). Statistical significance was analyzed using the Kruskal-Wallis 1-way ANOVA. At six days, cell viability was assessed with a fluorescent cytotoxicity kit (Molecular Probes, 2.0 mM calcein AM and 4.0 mM ethidium homodimer). Other cultures were fixed overnight in 4% paraformaldehyde and blocked with 10% normal goat serum or FBS containing 0.1% v/v Triton X-100 in PBS for one hour at room temperature. After rinsing, samples were stained with 6-diamidino-2-phenylindole (DAPI, 2 micrograms mL⁻¹, Roche) to visualize cell nuclei and with rhodamine-conjugated phalloidin (1:200 dilution, Invitrogen) to visualize F-actin. Fluorescent images were obtained with a confocal microscope (Leica SPE) and manually analyzed with ImageJ software (NIH) to determine spread cell area (n=37-83 cells per condition). Statistical significance was determined using the Mann-Whitney t test. At day six, an assay (Invitrogen) was used to assess metabolic activity. Briefly, alamarBlue reagent was added to the cells (50 micromolar), incubated at 37 degrees Celsius for 2 hours, and analyzed for fluorescence signal (n=3 independent samples for each condition). Statistical significance was deter-mined using the Mann-Whitney t test.

### Results

Engineered Protein Synthesis and Conjugation with Photoreactive Moieties

ELP (SEQ ID No: 4) was synthesized through recombinant expression from a commercially available Escherichia coli host and purified by utilizing the material's lower critical solution temperature (LCST). As with other elastin-like variants with the repetitive VPGXG (SEQ ID No: 2) amino acid sequence (where X is any amino acid), this engineered ELP is soluble in aqueous solutions below the LCST and forms a polymer-rich coacervate above this temperature. Therefore, engineered ELPs having canonical amino acids can be synthesized and purified using protocols that are easily scalable. The elastin-like repetitive sequence was modified to include one lysine residue per five VPGXG repeats to enable site-specific, post-purification reactivity through primary amine-based chemistry (full amino acid sequence given in FIG. 2). The purified ELP was covalently conjugated with a heterobifunctional NHS-ester diazirine photocrosslinker (succinimidyl 4,4′-azipentanoate), resulting in ELP-diazirine (ELP-D). The diazirine moiety is activated upon exposure to long wavelength ultraviolet light (330-370 nm), releasing N₂ and forming a highly reactive carbene intermediate that can rapidly insert into neighboring protein chains at any amino acid site (FIG. 1A).

The conjugation efficiency of the NHS-ester diazirine to the ELP was determined by quantifying the number of unreacted primary amines post-reaction. Three reaction times (6, 15, and 24 hours) and four reaction stoichiometries (0.25, 0.5, 1.0, and 2.0 moles of crosslinker per mole of primary amines contained within the ELP) were assessed. As expected, the conjugation efficiency was directly related to the stoichiometric ratio of crosslinker to primary amines (FIG. 1B). A reaction time of 6 hours was found to be sufficient, as further increasing the reaction time did not improve the conjugation efficiency. After 6 hours, a range of 9.96% to 90.3% of primary amines were reacted, corresponding to an average of 1.4 to 12.6 diazirines attached per protein chain. Having reliable control over the conjugation efficiency, and hence the maximum number of potential crosslinks formed per protein chain, enables direct tuning of the final biomaterial crosslinking density. In addition, having reliable control over the number of unmodified lysine residues enables potential future use of the remaining primary amine side chains as grafting sites for other molecules, such as growth factors, drugs, or peptides. Further experiments utilized a stoichiometric ratio of 1:1 crosslinker:primary amine (58.5% conjugation efficiency) to demonstrate stable biomaterial formation even when 40% of lysine residues are unmodified.

The photoactivation kinetics of the diazirine moiety on ELP-D were examined using Fourier transform infrared spectroscopy (FTIR) by monitoring the peak intensity area of the diazirine v₃ fundamental peak (1460 cm⁻¹) (FIG. 1C). Photoactivation of diazirine releases N₂, which does not absorb infrared irradiation due to its symmetry. Therefore, an increase in transmittance (i.e., decrease in peak intensity area) at the characteristic diazirine peak is directly correlated with the formation of a photoactivated moiety. The photoactivation followed first-order reaction kinetics with an exponential decay in diazirine concentration and a diazirine half-life (t_{1/2}) of 119 seconds. These data suggest that the degree of diazirine activation, and hence the crosslinking concentration within ELP-D, can be directly controlled by varying the ultraviolet exposure time.

### Versatile Photo-Processing of ELP-D Films and Scaffolds

To demonstrate the versatility of ELP-D, several biomaterial processing techniques were employed to create stable 2D thin films and 3D scaffolds. First, ELP-D films were drop cast directly onto a hydrophobic surface such as Parafilm, dried in air, crosslinked by ultraviolet light exposure, and then easily removed from the surface by peeling to create a free-standing film. A 10 mg film was measured to be 50 micrometer thick and was able to fully support a 4.5 g mass. Tensile tests were performed on these drop cast films in the dehydrated state. The Young's modulus of the dehydrated film was 250 MPa, and the film displayed little plasticity prior to fracture. These films verify the functionality of the photoreactive moiety to form a strong network of crosslinked protein, resulting in a solid, mechanically stable material. As drop casting results in films of variable thickness, we then explored the use of spin coating to create films of defined, uniform thickness.

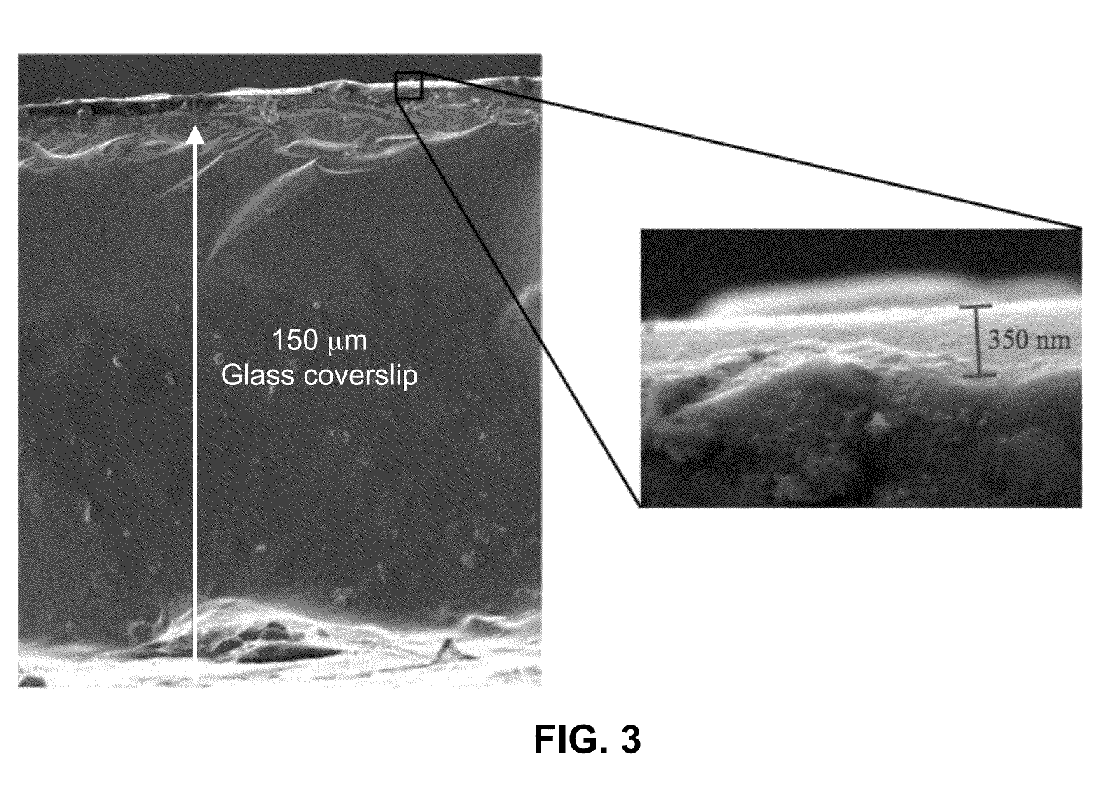

As a second demonstration, spin coating of ELP-D onto glass coverslips followed by photocrosslinking resulted in uniform protein coatings as was visualized by scanning electron microscopy. The retained protein mass, and hence thickness, of the films was controlled by altering the speed of the spin coating process. For a 70 microgram film, which corresponds to 10% protein retention during the spin coating process at a spin speed of 6000 rpm, the approximate thickness is estimated to be 500 nm (assuming a density of ELP-D on the order of native elastin, 1.3 mg cm⁻³). SEM of dried thin film cross-sections corroborated this estimation (FIG. 3). These thin films may be useful as resilient coatings on biomedical implants, where specific biofunctionality can be imparted to the implant surface through engineering of the modular engineered ELP.

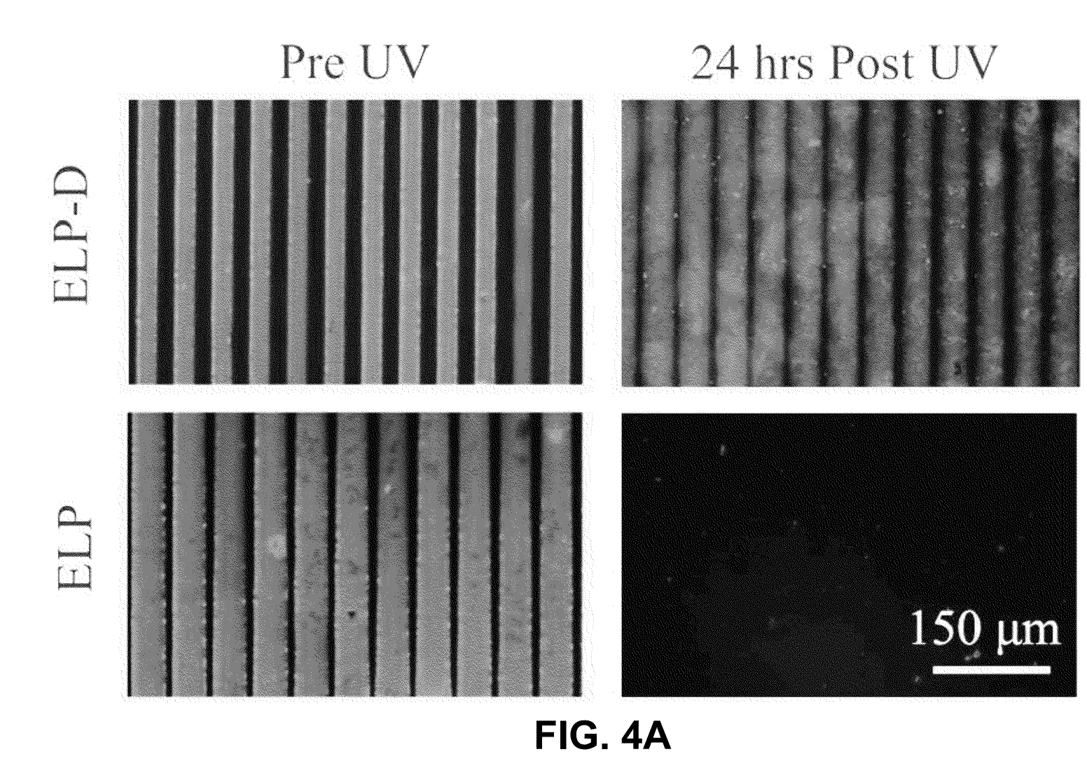

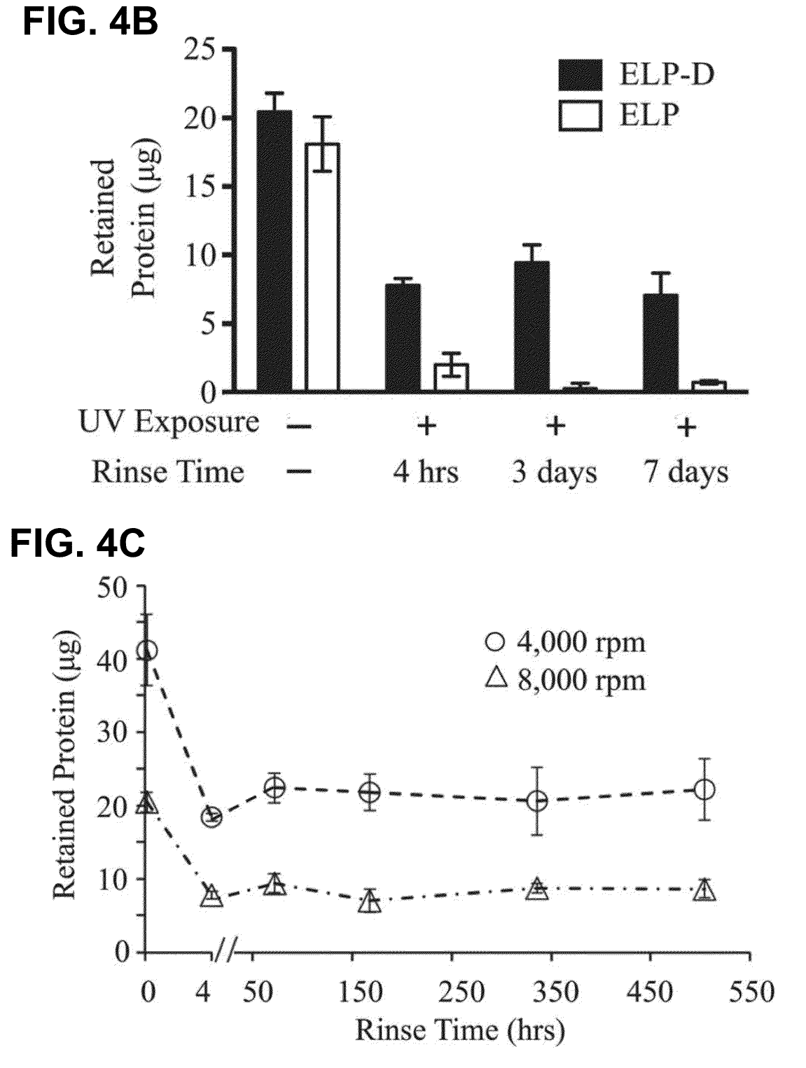

As another example, micropatterned topography was added to an ELP-D scaffold using soft lithography techniques. A poly-dimethylsiloxane (PDMS) stamp with parallel grooves, 50 micrometers wide and 5 micrometers deep, was used as a templating substrate for an ELP-D solution containing fluorescently labeled ELP-D for visualization. The templated and photocrosslinked ELP-D formed a scaffold with uniform and regularly spaced channels, as visualized by fluorescence microscopy (FIG. 4A). Soft lithography processing could be used to create large-scale, bioactive, patterned structures, promising for use as cell-directive scaffolds for engineering tissues where specific cellular orientation and organization are critical to function, such as in vascular and cardiac tissues.

The use of such a wide range of processing techniques exemplifies the versatility and amenability of ELP-D for a variety of biomaterial and tissue engineering applications. Unlike previous work that was limited to thin film applications, the scalable synthesis, purification, and photo-processing techniques utilized here enable the formation of 2D and 3D scaffolds ranging in size scale from nanometers to micrometers to millimeters.

### Photocrosslinked ELP-D Biomaterial Stability

A series of experiments were performed to assess the short- and long-term stability of photocrosslinked ELP-D materials. First, to demonstrate the requirement of the diazirine moiety, both ELP and ELP-D scaffolds were micropatterned on PDMS soft lithography molds. Prior to ultraviolet light exposure, fluorescence microscopy revealed that both materials formed micro-patterned surfaces with uniform grooves, although the raised ridges of ELP appeared slightly more swollen than ELP-D (FIG. 4A). Both patterned substrates were exposed to ultraviolet light for one hour followed by soaking in aqueous buffer for 24 hours. Further microscopy revealed that the ELP substrate was completely solubilized during this time, while the ELP-D material was still present and retained the striped micropattern, exhibiting swollen but still uniform grooves (FIG. 4A).

Next, to demonstrate thin film stability, solutions of ELP and ELP-D were spin coated onto glass coverslips. The amount of engineered protein retained on each coverslip was quantified before and at various time points after ultraviolet light exposure and rinsing (FIG. 4B). The ELP and ELP-D thin films initially contained a similar amount of material (˜19 micrograms, as shown by the no UV exposure and no rinsing condition). After ultraviolet light exposure and a four hour rinse, both films showed a loss of protein, with 38%+/−3.5% of ELP-D and 11%+/−4.7% of ELP retained on the coverslips. Over the subsequent week, protein retention on the ELP-coated cover-slips was negligible, while no further protein was lost from the ELP-D thin films. The initial ELP-D protein loss at four hours is presumably due to solubilization of protein chains that were not effectively crosslinked into the polymer network. Once these non-crosslinked chains were removed, the remainder of the ELP-D thin film retained its stability for up to one week (the longest time point tested).

Next, we assessed the role of initial thin film mass in determining the long-term film stability and required rinsing time. Glass coverslips were spin coated with ELP-D at either 8000 or 4000 rpm, yielding thin films with masses of ˜20 or ˜40 micrograms, respectively. These films were crosslinked by ultraviolet light exposure, and their protein retention was quantified over a three-week period of continuous rinsing (FIG. 4C). Both thin films experienced significant protein loss during the initial four-hour rinse. After this time point, both films proved to be very stable, losing negligible amounts of protein over the subsequent 21 days. Therefore, a four hour rinse time is sufficient to fabricate stable ELP-D thin films regardless of initial film mass. In addition, this protocol was found to be a highly reproducible method to produce thin films of a specific final mass, as evidenced by the narrow standard deviation for both sets of films. Due to this, we can reliably alter the final quantity of protein retained on the coated surface, and thus the total concentration of presented bioactive ligand.

Stability is an integral parameter dictating potential biomedical applications. The high level of stability displayed by photo-crosslinked ELP-D thin films makes them promising materials for use as implant coatings, which commonly need to remain intact over long periods of time. Native elastin is highly persistent in the body, although it can be rapidly degraded by the enzyme elastase.

A high level of film stability was demonstrated for ELP-D conjugated at ˜60% efficiency, leaving ˜5 unreacted primary amines per protein chain. These reactive groups could be used to tether various drugs or growth factors into the ELP-D thin film. Alternatively, ELP-D conjugated with more crosslinker per protein chain could be used to decrease the initial protein loss, increasing the concentration of bioactive domains present on the coated surface.

Photocrosslinked ELP-D Films Support hASC Culture

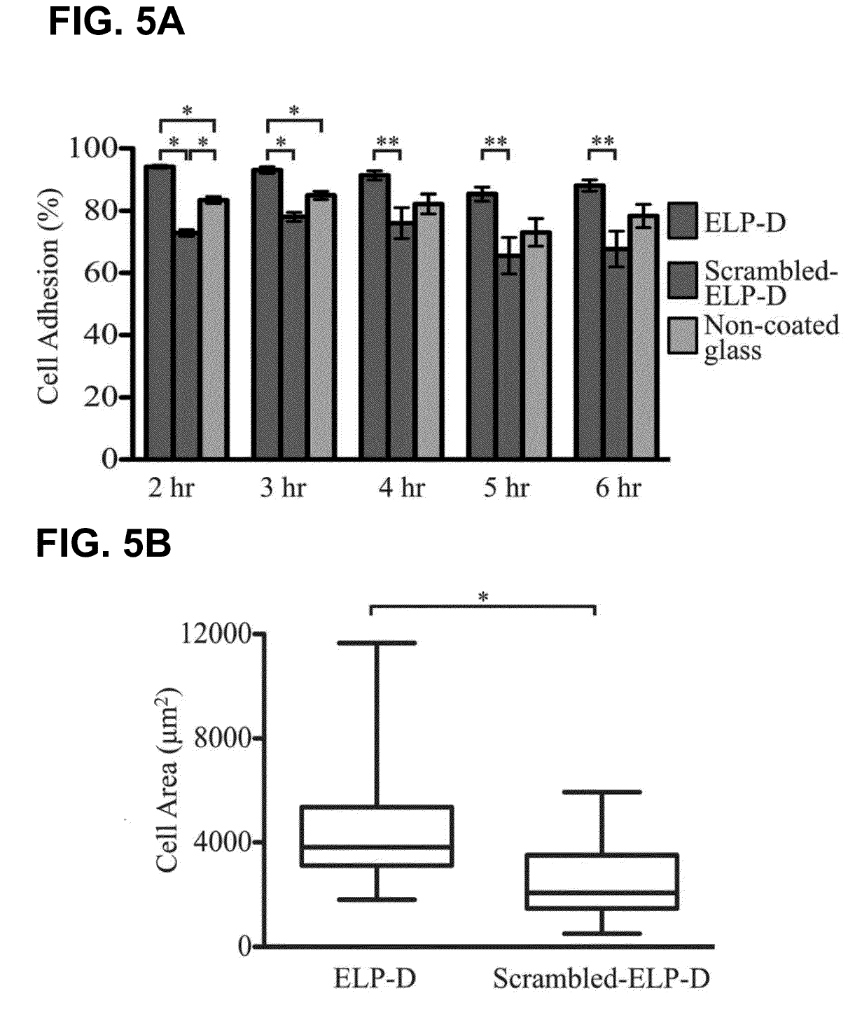

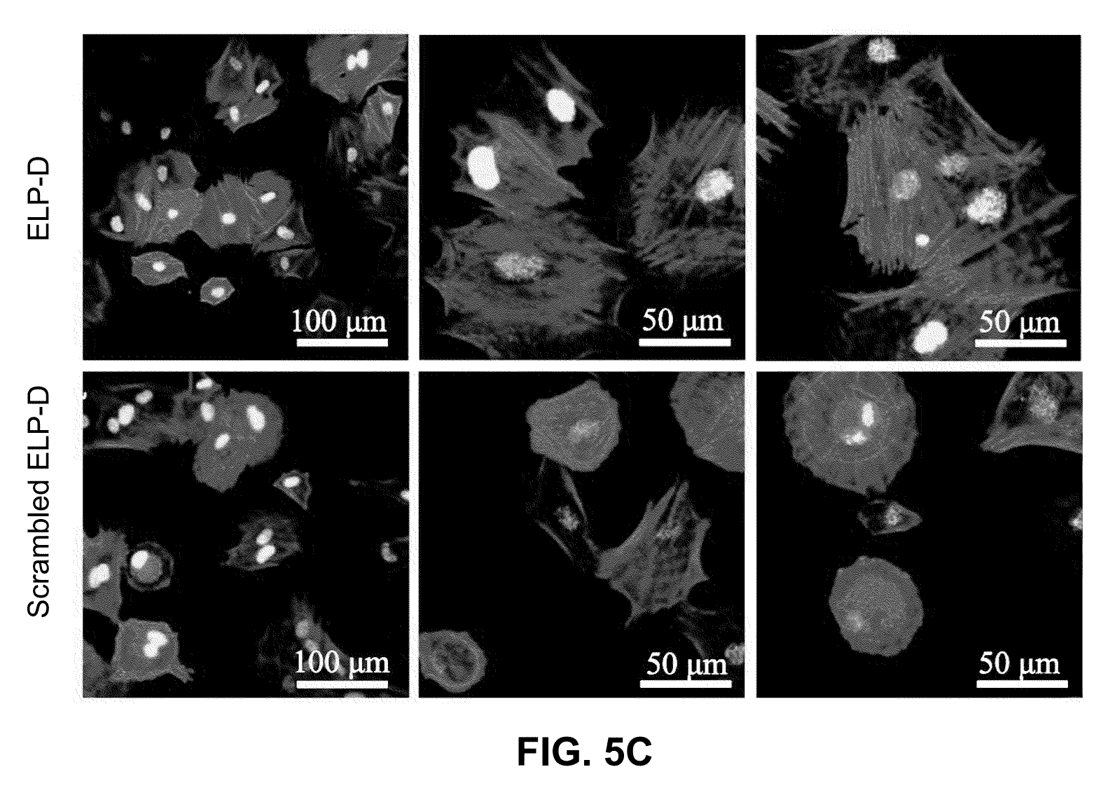

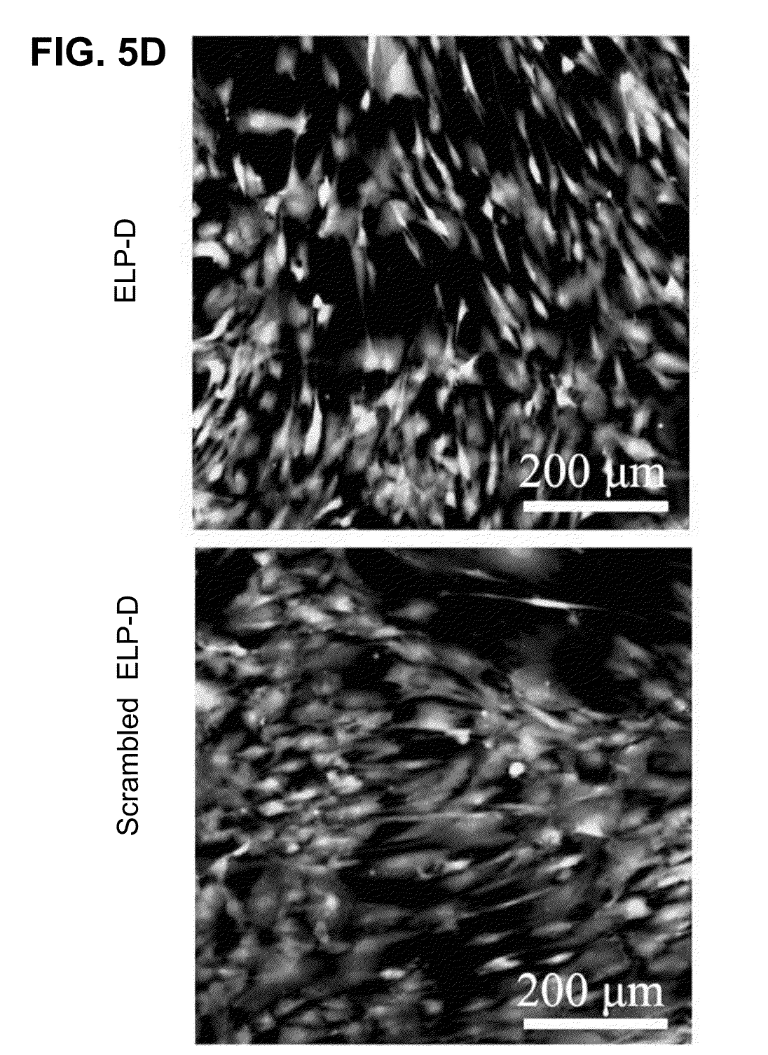

To demonstrate that the cell-adhesive, RGD bioactive domains retain functionality after photo-processing, human adipose-derived stem cells (hASCs) were seeded onto spin coated ELP-D thin films. hASCs are primary mesoderm cells isolated from adult tissue that can differentiate along any of the mesodermal lineages and can undergo osteogenic differentiation to become bone-forming osteoblast cells. For comparison, cells were seeded on non-coated glass as well as negative control thin films that were otherwise identical to ELP-D except they contained a non-cell-adhesive RDG peptide sequence (scrambled-ELP-D). All experiments were performed in medium supplemented with 10% serum. During the six hours immediately after seeding, a significantly larger percentage of hASCs were adherent on the ELP-D thin films compared to the scrambled-ELP-D surfaces, as manually quantified from phase contrast imaging (FIG. 5A).

To further investigate potential morphological differences between adherent cells on ELP-D and scrambled-ELP-D films, hASCs were fixed and fluorescently stained four hours after seeding. Individual cells were manually outlined, and cell spread areas were quantified using ImageJ software. Although the hASC population exhibited a wide, non-Gaussian distribution in spread area on both surfaces, the ELP-D films initiated significantly more spreading than the scrambled-ELP-D films (FIG. 5B). In addition, fluorescent micrographs of F-actin polymerized cytoskeletal fibers qualitatively corroborate these data (FIG. 5C).

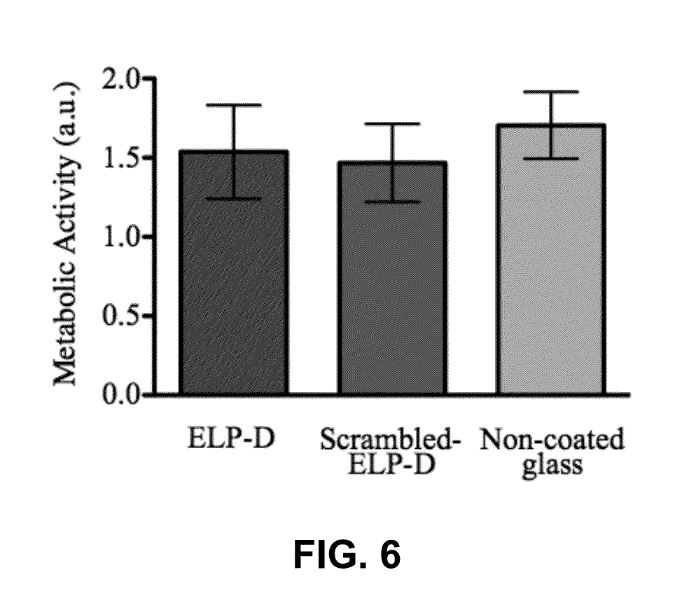

Cytocompatibility of the ELP-D thin films was confirmed through a longer, six-day study. As expected, fluorescent stains for live (calcein AM) and dead (ethidium homodimer) cells showed hASCs on all substrates (ELP-D, scrambled-ELP-D, and non-coated glass) were adherent and alive (with average viabilities of 98.9%+/−0.5%, 98.8%+/−0.3%, and 98.4%+/−0.7%, respectively). This result was expected, as ASCs are known to non-specifically adhere to many surfaces such as non-coated glass at longer time points. This cell adhesion is presumably due to the presence of ASC-secreted matrix proteins as well as serum proteins in the medium, which can adsorb to the substrate and induce cell adhesion. Additionally, quantification of cell metabolism using an assay found cells on all substrates to be similarly metabolically active at the six-day time point (FIG. 6).

These results validate the use of photo-processed ELP-D as a biomaterial, as it can elicit specific cell-matrix interactions and is cytocompatible. The increased adhesion rate of hASCs on ELP-D compared to scrambled-ELP-D films at early time points confirms that the RGD domain retains its cell-adhesive functionality even after photo-processing. As expected, at longer time points, hASC adhesion to the protein thin films is no longer dependent on RGD-cell interactions. The increased adhesion rate of hASCs on ELP-D thin films, even in a serum-rich environment, is promising for applications in which rapid cell adhesion is desired.

### ELP-D Coatings Significantly Improve the Adhesion of Bone-Forming Cells to Titanium Alloy

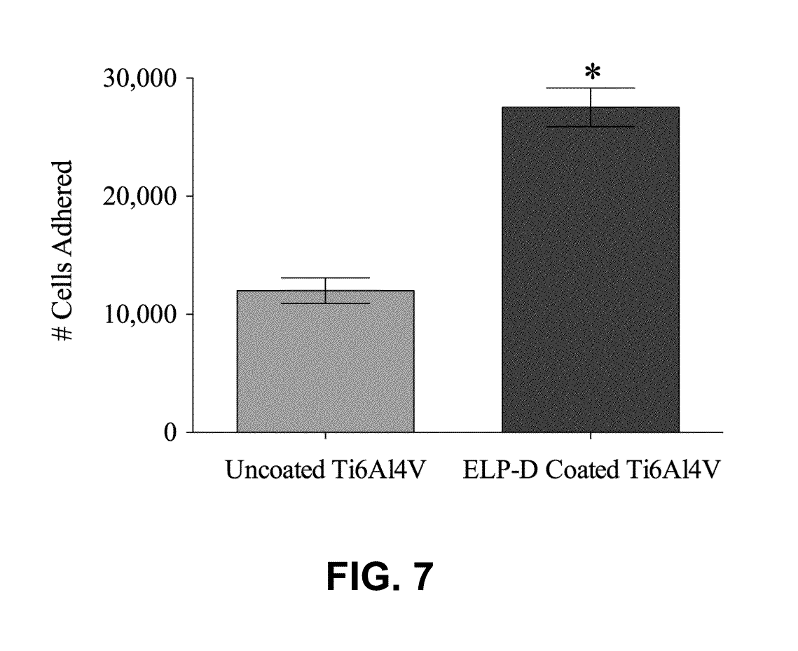

ELP-D coatings were produced on the titanium alloy Ti6Al4V, one of the most common materials used to make orthopedic implants. To assess the ability of the ELP-D coatings on this relevant substrate to encourage adhesion of bone-forming cells, MG63 cells, a human osteoblast-like cell line derived from osteosarcoma, were seeded onto both uncoated Ti6Al4V surfaces and ELP-D coated Ti6Al4V surfaces. After 24 hours, over twice as many MG63 cells had adhered to the ELP-D coated Ti6Al4V substrates compared with the uncoated Ti6Al4V. This demonstrated the cell-adhesive properties of the ELP-D coating on a material relevant to bone interfacing implants. (FIG. 7).

ELP-D Coatings Significantly Improve the Amount of Mineralization that Bone-Forming Cells Synthesize on Top of a Titanium Alloy Substrate

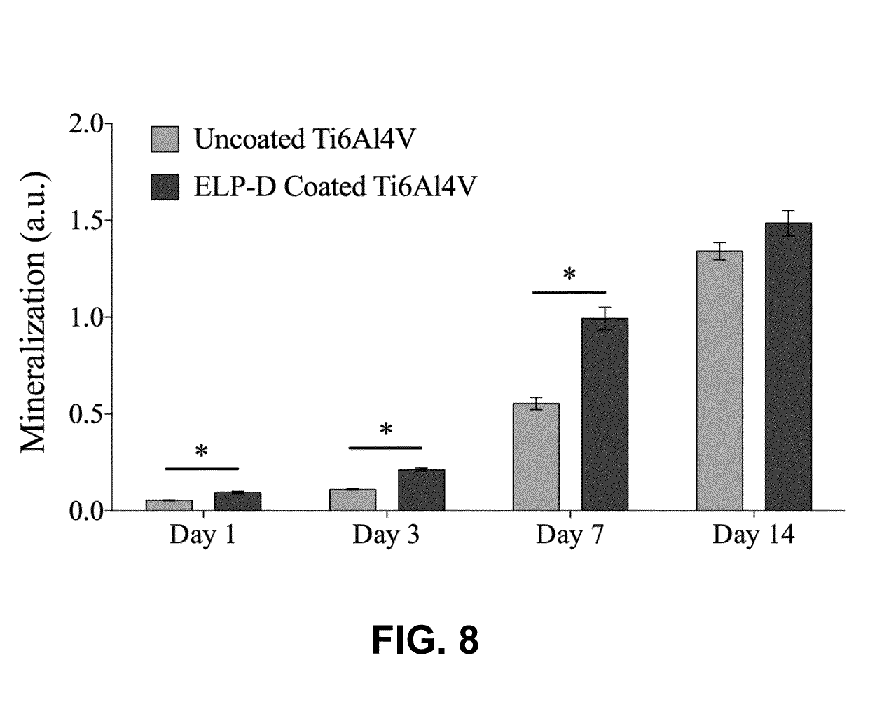

To assess the ability of bone-forming cells to produce mineralization, one component of new bone generation, on ELP-D coated Ti6Al4V compared with uncoated Ti6Al4V, MG63 cells were seeded on each substrate and allowed to adhere for 24 hours. At 24 hours, the cell culture media was replaced with fresh media that also contained 8 mM CaCl₂ (mineralization media). After 1, 3, 7, and 14 days in mineralization media, the quantity of produced mineralization was measured by staining the samples with alizarin red and solubilizing the stain with cetylpyridinium chloride. The mineral content in solution was measured spectroscopically. After 1, 3, and 7 days in mineralization media, the MG63 cells seeded onto ELP-D coated Ti6Al4V produced significantly more mineralization than cells seeded on uncoated Ti6Al4V. (FIG. 8).

ELP-D Coated Dental Screws Implanted into Rats Resulted in Faster Rates of Healing

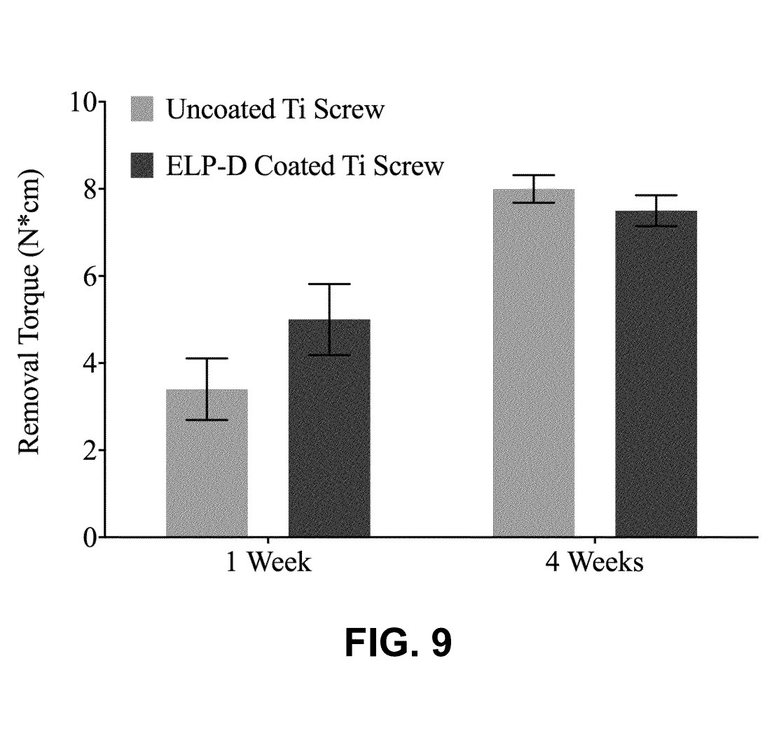

Commercially-pure titanium dental screws with and without ELP-D coatings were implanted into femurs of rats. At 1 and 4 weeks post-surgery, the degree of healing was characterized by measuring how much torque was needed to remove the screws from the femur. At 1 week, more torque was required to remove the ELP-D coated screws compared to the uncoated screws, suggesting that healing and osseointegration (integration between native bone and the implant) was quicker with the ELP-D coating (FIG. 9). Furthermore, at 4 weeks, the ELP-D coated and uncoated screws had similar removal torques, showing that the coating does not interfere with long-term healing.

### Photocrosslinked ELP-D Biomaterial Stability on Ti6Al4V

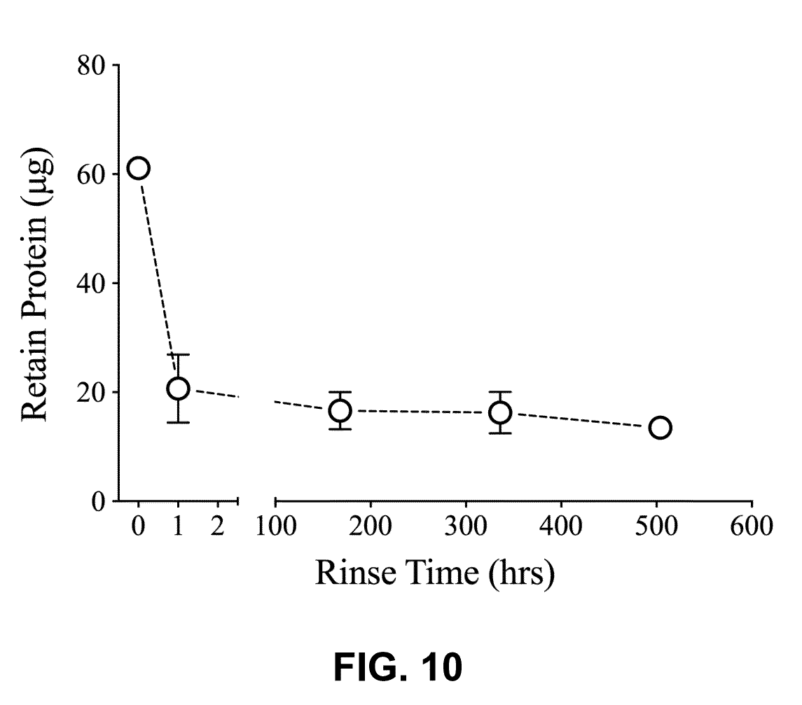

Coatings of ELP-D were produced on Ti6Al4V discs by spin coating. The coatings were made via a dual spin coating process, where 7 microliters of a 5 wt % ELP-D solution were added to the substrate and spun for 90 seconds at 4,000 rpm. The sample was then exposed to 365 nm UV light for 1 hour before a second coating was applied with the same parameters as above. The stability of the ELP-D coating on the Ti6Al4V was assessed with the same method as used above in FIG. 4B. The amount of protein on the substrate was quantified before and at various time points after exposure to UV light and rinsing in PBS. There was a loss of roughly 40 micrograms of protein during the first hour of rinsing but the mass of retained protein was not significantly changed as rinsing continued for three weeks, demonstrating the long-term stability of the ELP-D coating on Ti6Al4V (FIG. 10).

### Resilience of ELP-D Coating on Ti During Implantation/Explantation

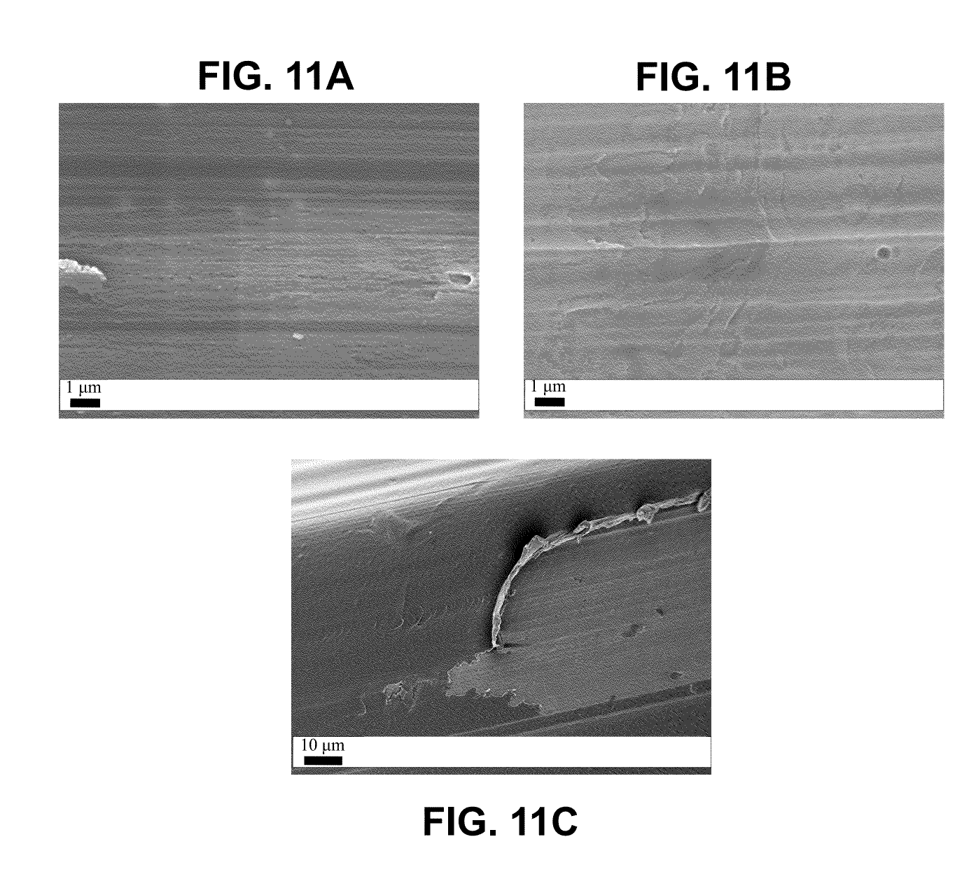

ELP-D coatings on commercially-pure titanium dental screws were fabricated by spin coating about 100 microliters of 5 wt % ELP-D solution onto the screws for 90 seconds at 4,000 rpm followed by exposure to 365 nm UV light for 1 hour. To demonstrate the resilience of the ELP-D coating needed to withstand the implantation process, ELP-D coated screws were screwed into and subsequently screwed out of a synthetic bone mimic (Sawbones, Vashon Island, Wash.) and observed under a scanning electron microscope. The ELP-D coating did not appear different between a coated screw that had not undergone implantation compared with a coated screw that had been implanted and explanted, with the ELP-D coating remaining intact over the majority of the screw surface area. One defect in the coating is also shown as a contrasting image to provide evidence that the coating was intact over the majority of the rest of the screw (FIGS. 11A-C).

### ELP-D Coating Topography on Ti6Al4V

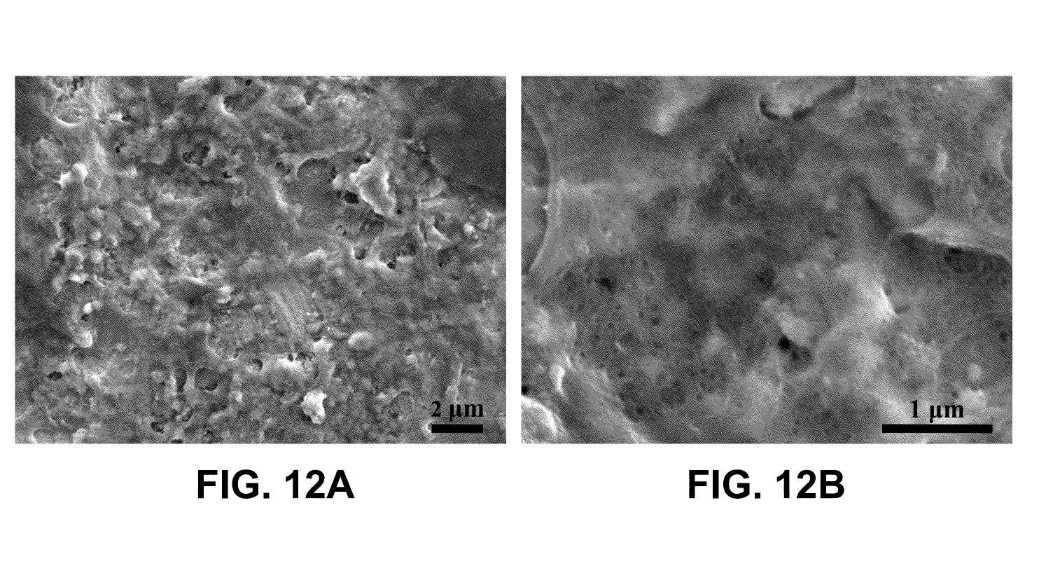

Spin coated ELP-D coatings on Ti6Al4V were imaged via scanning electron microscopy to show the coating topography. The ELP-D coating can be seen almost completely covering the rough Ti6Al4V substrate, the topographical features of which can be seen below the coating in certain areas (FIGS. 12A-B).

### Bone-Forming Cells Adhere and Spread on ELP-D Coated Ti6Al4V

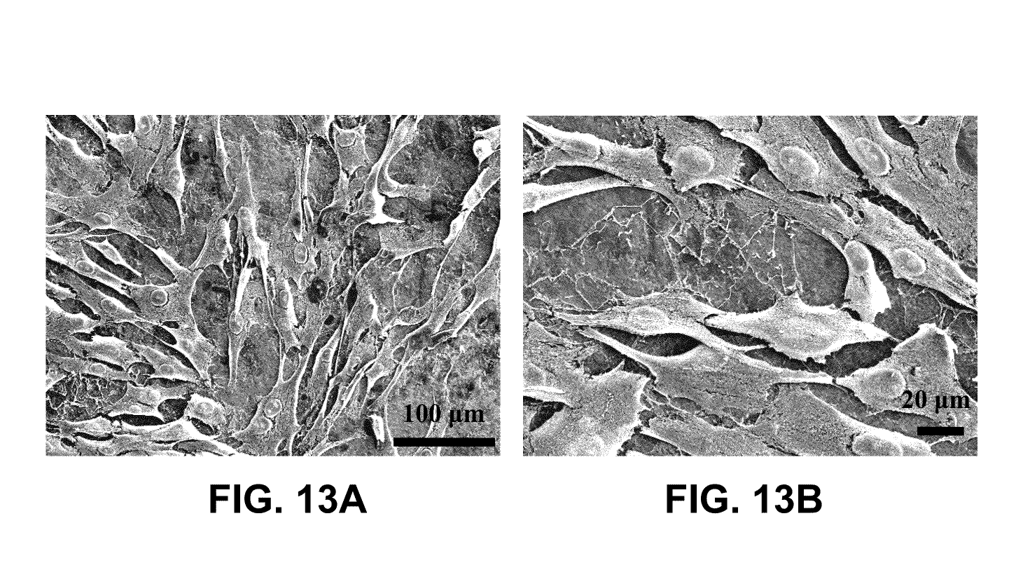

The adhesion and spreading of bone-forming MG63 cells seeded onto ELP-D coated Ti6Al4V was observed via scanning electron microscopy. The MG63 cells can be seen adopting a flattened, spread morphology with extended filopodia on the ELP-D coated Ti6Al4V surfaces (FIGS. 13A-B).

The cell-adhesive domain could have functional variants from sequences like TVYAVTGRGDSPASSAA (SEQ ID No: 3) as long as they contain an RGD sequence.

Elastin-like sequence listing:

(SEQ ID No: 2)
TVYAVTGRGDSPASSAASA VPGIG VPGIG VPGKG VPGIG VPGIG
VPGIG VPGIG VPGKG VPGIG VPGIG VPGIG VPGIG VPGKG
VPGIG VPGIG
(SEQ ID No: 13)
TVYAVTGRGDSPASSAASA VPGIG VPGIG VPGKG VPGIG VPGIG
(SEQ ID No: 14)
TVYAVTGRGDSPASSAASA VPGIG VPGIG VPGKG VPGIG VPGIG
VPGIG VPGIG VPGKG VPGIG VPGIG VPGIG VPGIG VPGKG
VPGIG VPGIG VPGIG VPGIG VPGKG VPGIG VPGIG VPGIG
VPGIG VPGKG VPGIG VPGIG
(SEQ ID No: 15)
(TVYAVTGRGDSPASSAASA VPGIG VPGIG VPGKG VPGIG
VPGIG VPGIG VPGIG VPGKG VPGIG VPGIG VPGIG VPGIG
VPGKG VPGIG VPGIG)₂
(SEQ ID No: 16)
(TVYAVTGRGDSPASSAASA VPGIG VPGIG VPGKG VPGIG
VPGIG VPGIG VPGIG VPGKG VPGIG VPGIG VPGIG VPGIG
VPGKG VPGIG VPGIG)₄
(SEQ ID No: 17)
(TVYAVTGRGDSPASSAASA VPGIG VPGIG VPGKG VPGIG
VPGIG VPGIG VPGIG VPGKG VPGIG VPGIG VPGIG VPGIG
VPGKG VPGIG VPGIG)₈
(SEQ ID No: 18)
ASSAASGRGDSPASSAASA VPGIG VPGIG VPGKG VPGIG VPGIG
VPGIG VPGIG VPGKG VPGIG VPGIG VPGIG VPGIG VPGKG
VPGIG VPGIG
(SEQ ID No: 19)
GGSGGSGGRGDSGGSGGS VPGIG VPGIG VPGKG VPGIG VPGIG
VPGIG VPGIG VPGKG VPGIG VPGIG VPGIG VPGIG VPGKG
VPGIG VPGIG
(SEQ ID No: 20)
TVYAVTGRGDSPASSAASA VPGVG VPGVG VPGKG VPGVG VPGVG
VPGVG VPGVG VPGKG VPGVG VPGVG VPGVG VPGVG VPGKG
VPGVG VPGVG
(SEQ ID No: 21)
TVYAVTGRGDSPASSAASA VPGIG VPGVG VPGKG VPGIG VPGVG
VPGIG VPGVG VPGKG VPGIG VPGVG VPGIG VPGVG VPGKG
VPGIG VPGVG
(SEQ ID No: 22)
TVYAVTGRGDSPASSAASA VPGKG VPGIG VPGKG VPGIG VPGKG
VPGIG VPGKG VPGIG VPGKG VPGIG VPGKG VPGIG VPGKG
VPGIG VPGKG
(SEQ ID No: 23)
TVYAVTGRGDSPASSAASA VPGIG VPGKG VPGKG VPGIG VPGKG
VPGKG VPGIG VPGKG VPGKG VPGIG VPGKG VPGKG VPGIG
VPGKG VPGKG
(SEQ ID No: 24)
TVYAVTGRGDSPASSAASA VPGVG VPGKG VPGKG VPGVG VPGKG
VPGKG VPGVG VPGKG VPGKG VPGVG VPGKG VPGKG VPGVG
VPGKG VPGKG
(SEQ ID No: 25)
TVYAVTGRGDSPASSAASA VPGKG VPGKG VPGKG VPGKG VPGKG
VPGKG VPGKG VPGKG VPGKG VPGKG VPGKG VPGKG VPGKG
VPGKG VPGKG
(SEQ ID No: 26)
TVYAVTGRGDSPASSAASA VPGIG VPGIG VPGIG VPGIG VPGKG
VPGIG VPGIG VPGIG VPGIG VPGKG VPGIG VPGIG VPGIG
VPGIG VPGIG

What is claimed is:

1. A bone interfacing medical implant, comprising:
(a) a bone interfacing coating for promoting bone regeneration, wherein the coating is an engineered protein containing an elastin-like structural domain (SEQ ID No: 2) and a cell-adhesive domain derived from an extended fibronectin RGD sequence; and
(b) a medical implant with its surface covalently and directly bonded to the coating via photoreactive crosslinking through an insertion or an addition reaction.

2. The bone interfacing medical implant as set forth in claim 1, wherein the elastin-like structural domain comprises a repetitive amino acid sequence of the form (VPGXG)ₙ (SEQ ID No: 2), where X is an amino acid and n is at least 5.

3. The bone interfacing medical implant as set forth in claim 2, wherein at least 5% of X residues are K amino acids.

4. The bone interfacing medical implant as set forth in claim 1, wherein the extended fibronectin cell-binding sequence is TVYAVTGRGDSPASSAA (SEQ ID No: 3).

5. The bone interfacing medical implant as set forth in claim 1, wherein the cell-adhesive domain comprises at least a RGD sequence.

6. The bone interfacing medical implant as set forth in claim 1, wherein the surface is covalently and directly bonded to the coating via a heterobifunctional, photoreactive crosslinker with activated carbene or nitrene groups.

7. The bone interfacing medical implant as set forth in claim 1, wherein the photoreactive crosslinking is through an insertion reaction of carbene groups, an insertion reaction of nitrene groups, an addition reaction with free radicals, or a multi-step insertion and addition reaction of carbene or nitrene groups.

8. The bone interfacing medical implant as set forth in claim 1, wherein the medical implant is a joint replacement, a dental implant, a bone screw, or a bone interfacing device.

9. The bone interfacing medical implant as set forth in claim 1, wherein at least the surface of the medical implant comprises titanium, titanium alloy, stainless steel, ceramic, tantalum, tantalum alloy, cobalt-chromium alloy, magnesium alloy, alumina, zirconia, zirconia toughened alumina, hydroxyapatite/calcium phosphates, or polyether ether ketone.
